(19) United States
(12) Patent Application Publication
(10) Pub. No.: US 2022/0003756 A1
(43) Pub. Date: Jan. 6, 2022

(54) METHODS FOR BONDING MOLECULES TO RUTHENIUM SURFACES

(71) Applicant: Roswell Biotechnologies, Inc., San Diego, CA (US)

(72) Inventor: Brandon Wenning, San Diego, CA (US)

(73) Assignee: Roswell Biotechnologies, Inc., San Diego, CA (US)

(21) Appl. No.: 17/354,900

(22) Filed: Jun. 22, 2021

Related U.S. Application Data
(60) Provisional application No. 63/042,487, filed on Jun. 22, 2020.

(51) Int. Cl. G01N 33/543

(52) U.S. Cl. CPC G01N 33/5438

(57) ABSTRACT

A bifunctional linker of general structure A-L-Z is used to covalently bond a bridge molecule to ruthenium electrodes in sensor circuits. The A group comprises a diazonium salt, a diazo group or a carbene precursor such as an imidazolium ring. L is a bivalent tether configured to adjust the spacing of Z from the ruthenium surface and to alter conductivity through the circuit. An end of the bridge molecule to be bonded to ruthenium through the linker is configured with a functional group that participates in a condensation reaction or click-chemistry with the Z group of the bifunctional linker.

The instant disclosure claims the filing date priority benefit of Provisional Application Ser. No. 63/042,487, filed Jun. 22, 2021; the disclosure of which is incorporated herein in its entirety.

### FIELD

The present disclosure generally relates to functionalization of metal surfaces and in particular to methods to covalently bond molecules to ruthenium surfaces.

### BACKGROUND

Molecular sensors by definition include a molecule as an essential element of the sensor. In some examples, a sensor includes a molecule attached in some way to a metal electrode, and in specific cases, a sensor comprises a single molecule called a “bridge molecule” bridged across a gap between spaced-apart metal electrodes. Such sensors may require specific binding of a first end of a bridge molecule to a first electrode in a pair of electrodes and binding of a second end of the bridge molecule to a second electrode in the pair of electrodes, such that the single bridge molecule bridges the gap between spaced-apart electrodes and closes an otherwise open circuit. Making sensors of this configuration is exceedingly intricate and complicated, requiring formation of stable metal-bridge bonds capable of conducting electrical current, in a controlled assembly process, e.g., self-assembly, that ensures bridge molecules bridge across spaced-apart electrodes to close a circuit, rather than bond to single electrodes to form unproductive and non-conducting loops.

Various metals may be envisioned for electrodes in sensor circuits, such as, for example, gold (Au), silver (Ag), platinum (Pt), palladium (Pd), titanium (Ti), nickel (Ni), ruthenium (Ru), aluminum (Al), and copper (Cu), circuitry production in semiconductor chip foundries negates use of Au, forcing some innovators to turn to other metals. Although a molecule having a thiol (—SH) group may be bonded to Au by formation of a thiol-Au bond, many other metals cannot readily form such bonds. Alternative molecule-metal bonds may be far from covalent, and may be unstable and not particularly conductive to current flow between metal and molecule.

Ruthenium is a particularly favorable metal to use in molecular sensors because it can be readily deposited on substrates by various lithographic methods and it can be used in semiconductor chip foundries. Further, bonding and coordination to ruthenium are at least somewhat known, (see, for example: L. M. Martinez-Prieto, et al., “Organometallic Ruthenium Nanoparticles: Synthesis, Surface Chemistry, and Insights into Ligand Coordination,” Acc. Chem. Res., 2018, 51, 376-384; M. P. Stewart, et al., “Direct Covalent Grafting of Conjugated Molecules onto Si, GaAs, and Pd Surfaces from Aryldiazonium Salts,” J. Am. Chem. Soc., 2004 126, 370-378; G. S. Tulevski, et al., “Formation of Catalytic Metal-Molecule Contacts,” Science, 2005, 309, 591-594; and F. Ren, et al., “Polymer Growth by Functionalized Ruthenium Nanoparticles,” Macromolecules, 2007, 40, 8151-8155.

However, methods for covalently bonding molecules to ruthenium surfaces are needed if innovators are to use ruthenium electrodes in molecular sensors. Ruthenium presents unique challenges for surface functionalization, and many methods from other materials cannot be applied to these electrodes, presenting a need for alternate synthetic strategies to modify the surface. Further, regioselective synthetic methods are needed so that a single bridge molecule can be selectively and covalently bonded to spaced-apart ruthenium electrodes directionally, to form a stable and conducting closed sensor circuit.

### SUMMARY

In accordance with various embodiments of the present disclosure, covalent chemical functionalization or passivation of ruthenium surfaces is carried out through a range of functional molecules, including aryl diazonium salts, organic diazo compounds, and N-heterocyclic carbenes, which react with the ruthenium surface forming a stable organic passivation layer.

In various embodiments, methods for bonding molecules to ruthenium surfaces are described. In various embodiments, a molecule is bonded to a ruthenium surface through a bifunctional linker having a first end configured to covalently bond to the ruthenium and a second end configured to bond to the molecule.

In various embodiments, two uniquely configured bifunctional linkers are used to covalently bond each end of a bridge molecule to spaced-apart ruthenium electrodes to close an otherwise open sensor circuit.

In various embodiments, a sensor circuit comprises: a pair of ruthenium electrodes comprising a first ruthenium electrode and a second ruthenium electrode spaced-apart from the first ruthenium electrode by a nanogap; and a bridge molecule comprising a first reactive group RG_{A} configured at or near a first end, and a second reactive group RG_{B} configured at a second end, the bridge molecule electrically wired to each of the first and second ruthenium electrodes and spanning the nanogap; wherein the first reactive group RG_{A} is conjugated to a first reactive group Z¹ covalently bonded to the first ruthenium electrode through a first bivalent tether L, and the second reactive group RG_{B} is conjugated to a second reactive group Z² covalently bonded to the second ruthenium electrode through a second bivalent tether L′.

In various embodiments, the bridge molecule comprises a polypeptide, a protein, a protein fragment, a protein alpha-helix, DNA, RNA, a single-stranded oligonucleotide, a double-stranded oligonucleotide, a peptide nucleic acid duplex, a peptide nucleic acid-DNA hybrid duplex, an antibody, an antibody Fab binding domain, a carbon nanotube, a graphene-like polycyclic aromatic nanoribbon, other natural polymers, or (poly)thiophene.

In various embodiments, RG_{A} and RG_{B} are independently selected from —CO₂H, —NH₂, —OH, —SH, —CH═CH₂, —C≡CH, and —N₃.

In various embodiments, L further comprises a phenyl ring or substituted phenyl ring covalently bonded to the first ruthenium electrode.

In various embodiments, L′ further comprises a phenyl ring or substituted phenyl ring covalently bonded to the second ruthenium electrode.

In various embodiments, a bifunctional linker configured to covalently bond a molecule to a ruthenium surface has the structure A-L-Z, wherein:

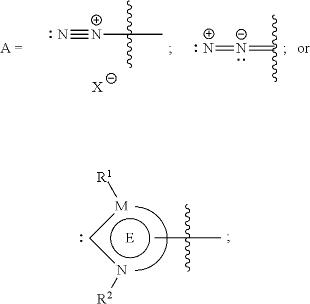

X=Cl—, Br—, I—, BF₄—, ClO₄—, or (SO₄²⁻)_{1/2};
Z=—CO₂H, —NH₂, —OH, —OC(O)C(CH₃)₂—Br, —CH═CH₂, —SH, —C≡CH or N₃;
L is a bivalent tether selected from -G-CH—; -G-(CH₂)_{y}—; or -G-(CH₂CH₂O)_{y}—, wherein y=1 to 25 and G is an optional aryl linkage —Ar—;
M=N or S, and E is a heterocycle selected from imidazole, imidazoline, thiazole, or triazole; and
R¹ and R² are independently selected from an electron pair, H, an aliphatic substituent, or an aryl substituent.

In various embodiments, A is a diazonium salt and Z is —CO₂H, —NH₂, —OH, —OC(O)C(CH₃)₂—Br, —CH═CH₂, —SH, —C≡CH or N₃.

In various embodiments, A is a diazo group and Z is —CO₂H, —NH₂, —OH, —OC(O)C(CH₃)₂—Br, —CH═CH₂, —SH, —C≡CH or N₃.

In various embodiments, A is an imidazolium ring and Z is —CO₂H, —NH₂, —OH, —OC(O)C(CH₃)₂—Br, —CH═CH₂, —SH, —C≡CH or N₃.

In various embodiments, a method of forming a sensor circuit, the method comprises: depositing a pair of ruthenium electrodes on a substrate, the pair of ruthenium electrodes comprising a first ruthenium electrode and a second ruthenium electrode spaced-apart from the first ruthenium electrode by a nanogap; exposing the first ruthenium electrode to a bifunctional linker having a structure A-L-Z¹ to functionalize the first ruthenium electrode with a plurality of exposed Z¹ groups; conjugating at least one exposed Z¹ group to a first reactive group RG_{A} configured at or near a first end of a bridge molecule, the bridge molecule further comprising a second reactive group RG_{B} configured at a second end of the bridge molecule; exposing the second ruthenium electrode to a bifunctional linker having a structure A′-L′-Z² to functionalize the second ruthenium electrode with a plurality of exposed Z² groups; and conjugating at least one exposed Z² group to the second reactive group RG_{B} configured at or near the second end of the bridge molecule.

In various embodiments, A and A′ are independently,

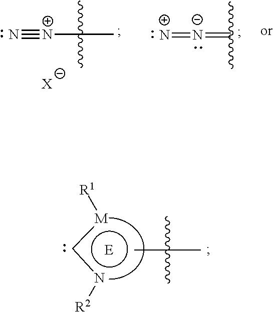

X=Cl—, Br—, I—, BF₄—, ClO₄−, or (SO₄²⁻)_{1/2};
Z¹ and Z² are independently selected from:
—CO₂H, —NH₂, —OH, —OC(O)C(CH₃)₂—Br, —CH═CH₂, —SH, —C≡CH, and N₃;
L and L′ are each a bivalent tether independently selected from -G-CH—; -G-(CH₂)_{y}—; or -G-(CH₂CH₂O)_{y}—, wherein y=1 to 25 and G is an optional aryl linkage —Ar—;
M=N or S, and E is a heterocycle selected from imidazole, imidazoline, thiazole, or triazole; and
R¹ and R² are independently selected from an electron pair, H, an aliphatic substituent, or an aryl substituent.

In various embodiments, a method of forming a sensor circuit further comprises a step of polarizing the first ruthenium electrode prior to exposing the first ruthenium electrode to a bifunctional linker having a structure A-L-Z¹ such that the bifunctional linker covalently bonds to the first ruthenium electrode via electrochemical reduction.

In various embodiments, a method of forming a sensor circuit further comprises a step of polarizing the second ruthenium electrode prior to exposing the second ruthenium electrode to a bifunctional linker having a structure A′-L′-Z² such that the bifunctional linker covalently bonds to the first ruthenium electrode via electrochemical reduction.

In various embodiments of a method of forming a sensor circuit, the bridge molecule comprises a polypeptide, and RG_{A} and RG_{B} are independently selected from —CO₂H, —NH₂, —OH, —SH, —CH═CH₂, —C≡CH, and —N₃.

### BRIEF DESCRIPTION OF THE DRAWING FIGURES

The subject matter is pointed out with particularity and claimed distinctly in the concluding portion of the specification. A more complete understanding, however, may best be obtained by referring to the detailed description and claims when considered in connection with the following drawing figures:

FIG. 1 sets forth a general method for closing an open sensor circuit by bridging a bridge molecule to functionalized ruthenium electrodes, in accordance with various embodiments of the present disclosure;

FIGS. 2A-2B illustrates a series of synthetic organic transformations usable to conjugate a molecule to a bifunctional linker covalently bonded to a ruthenium surface (—Ru—);

FIG. 3 sets forth water contact angle measurements of ruthenium surfaces before and after covalent functionalization; and

FIG. 4 sets forth a bar chart showing changing in contact angle for no functionalization versus covalent functionalization for different pre-treatment conditions.

### DETAILED DESCRIPTION

The detailed description of exemplary embodiments makes reference to the accompanying drawings, which show exemplary embodiments by way of illustration and their best mode. While these exemplary embodiments are described in sufficient detail to enable those skilled in the art to practice the invention, it should be understood that other embodiments may be realized and that logical, chemical, and mechanical changes may be made without departing from the spirit and scope of the inventions. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

In various embodiments of the present disclosure, methods for bonding molecules to ruthenium surfaces are described. In various embodiments, a molecule is bonded to a ruthenium surface through a bifunctional linker having a first end configured to covalently bond to the ruthenium surface, and a second end configured to bond to the molecule.

### Definitions and Interpretations

As used herein, the abbreviation “—Ru—” refers to a portion of a ruthenium surface where covalent bonding is to occur. A ruthenium surface herein may be a portion of a ruthenium electrode.

As used herein, the term “electrode” means any structure that can act as an efficient source or sink of charge carriers. Electrodes herein are metal or semiconductor structures, such as those used in electronic circuits. A pair of spaced-apart electrodes herein for a molecular sensor circuit may comprise a source and drain electrode pair, with the distance between spaced-apart electrodes in any one pair of electrodes referred to as a “nanogap.” The present disclosure is primarily concerned with ruthenium electrodes and the binding of molecules to the ruthenium surfaces, although there could be an extension of the bifunctional linkers and methods of binding molecules to other metals besides ruthenium.

As used herein, the term “bridge molecule” refers to a molecule having at least some electrical conductivity, i.e., a molecule capable of functioning as a “molecular wire,” functionalized at two distinct sites so that the bridge molecule can be wired into a circuit by conjugating each of the two distinct sites to each electrode in a pair of spaced-apart electrodes. Stated another way, a bridge molecule herein is configured to close an otherwise open circuit by conjugation of a first functionalized site on the bridge molecule to a first electrode and conjugation of a second functionalized site on the bridge molecule to a second electrode that is spaced-apart from the first electrode, with the closed circuit including a conductive pathway through the bridge molecule between the first and second functionalized sites. A bridge molecule herein may have a length of from about 1 nm to about 1 μm, depending on the size of the gap between spaced-apart electrodes that the bridge molecule is intended to span. Bridge molecules herein include, but are not limited to, polypeptides, proteins, protein fragments, protein alpha-helix, DNA, RNA, single-stranded oligonucleotides, double-stranded oligonucleotides, peptide nucleic acid duplex, peptide nucleic acid-DNA hybrid duplex, an antibody, an antibody Fab binding domain, a carbon nanotube, a graphene-like polycyclic aromatic nanoribbon, other natural polymers, or a synthetic polymer such as a (poly)thiophene. Bridge molecules for use herein may have a linear polymer structure or may be more “globular” in shape, such as through secondary and tertiary structures. In various embodiments, a bridge molecule herein is an association of at least two molecules, such as in the case of a bridge molecule comprising a protein having at least two subunits.

As used herein, the term “conjugation” or “bonding” refers to any of the wide variety of methods to physically attach one molecule to another, or a molecule to a metal surface or particle. Such methods typically involve forming covalent or non-covalent chemical bonds (ionic, H-bonding), but may also rely on protein-protein interactions, protein-metal interactions, or chemical or physical adsorption via intermolecular (e.g., Van der Waals) forces. Although there exists a large variety of such methods known to those skilled in the art of conjugation and organic chemistry, the present disclosure is primarily concerned with covalent bonding between carbon radicals, cations, or carbenes to ruthenium, and the covalent bonding characterized in the condensation of amines and carboxylic acids to produce amides, alcohols and carboxylic acids to produce esters, alcohols (or alkoxides) and halides (or tosylates, etc.) to produce ethers, carbon free radicals and alkenes to produce alkanes, and azides and alkynes to produce triazoles.

As used herein, the term “synthetic” in reference to a molecule such as a polymeric bridge molecule should not be construed to mean the entire molecule needs to be synthetically prepared. Further, as used herein, the term “bio” or “natural” in reference to a molecule such as a polymeric bridge molecule should not be construed to mean the entire molecule needs to be found in nature. Certainly, there is overlap, and synthetic molecules may incorporate portions of naturally occurring molecules, and vice versa. One non-limiting example is a bridge molecule for binding to ruthenium electrodes herein comprising a central portion of a naturally occurring polypeptide or naturally occurring oligonucleotide but where the ends of the natural polymer are capped with synthetically obtained functionality so that the molecule can bind to the bifunctional linkers described herein. These end caps may comprise phenyl groups with a diazonium salt substituent, or a N-heterocycle such as an imidazole. In another non-limiting example, a bridge molecule having a central core consisting of (poly)2,5-thiophene may be capped with amino acids or peptides, and the terminal amino acid or peptide may be the functional group used to bond to one of the bifunctional linkers described herein.

As used herein, the term “protecting group” takes on its ordinary meaning in synthetic organic chemistry. In general, a protecting group masks reactive functionality so that other reactions can be performed elsewhere on a molecule. Deprotection then reveals the original reactive functionality. A general resource for protecting groups and their use is Peter G. M. Wuts, “Greene's Protective Groups in Organic Synthesis,” 5^{th} ed., John Wiley & Sons, New York, N.Y., 2014, ISBN: 978-1-118-05748-3. Herein, the abbreviation “—P” may be used to show a protecting group, which should not be confused with a phosphorous atom. For example, a carboxylic acid represented as R—CO₂—P is protected, whereas the corresponding carboxylic acid R—CO₂H or R—CO₂ is unprotected.

### General Embodiments

In various embodiments, methods for covalently bonding molecules to ruthenium surfaces are provided. In various embodiments, a molecule is covalently bonded to a ruthenium surface by a bifunctional linker. In various embodiments, the molecule comprises a bridge molecule for a molecular sensor circuit.

In various embodiments, a bifunctional linker in accordance with the present disclosure comprises a first end (the “A-end”) configured to covalently bond to a ruthenium surface, and a second end (the “Z-end”) configured to covalently bond to a molecule. Configured in this manner, the bifunctional linker provides a way to bond the molecule to the ruthenium metal. In various embodiments, the molecule comprises a bridge molecule for a molecular sensor circuit.

In various embodiments, either or both of the first and second ends of the bifunctional linker may be protected, or provided in an inactivated state, such that a later deprotection step, or a later activation step, reconfigures an end of the bifunctional linker for covalent bonding to the ruthenium surface and/or to the molecule, as needed. In various embodiments, the molecule comprises a bridge molecule for a molecular sensor circuit. In various embodiments, a stepwise deprotection strategy is used to selectively bond each end of the bridge molecule to each electrode in a pair of electrodes.

In various embodiments, a ruthenium surface for functionalization with a molecule is part of a sensor circuit comprising ruthenium electrodes.

In various embodiments, an open sensor circuit comprises a pair of ruthenium electrodes, the pair of ruthenium electrodes further comprising a first ruthenium electrode and a second ruthenium electrode spaced apart from the first ruthenium electrode by a nanogap.

In various embodiments, a molecule to be bonded to one or more ruthenium electrodes comprises a bridge molecule further comprising a first functional site and a second functional site. In various embodiments, the bridge molecule comprises a biopolymer or a synthetic organic polymer having a first end and a second end. In various embodiments, the first and second functional sites of the bridge molecule reside near the first and second ends of a biopolymer or synthetic organic polymer.

In various embodiments, an open sensor circuit is closed by a bridge molecule, the bridge molecule having a first functional site at a first end and a second functional site at a second end, the circuit being closed by the bonding of the first functional site to a first ruthenium electrode previously passivated with a first bifunctional linker and the bonding of the second functional site to a second ruthenium electrode, spaced-apart from the first ruthenium electrode, wherein the second ruthenium electrode was previously passivated with a second bifunctional linker.

In various embodiments, the structure of the bifunctional linker affects the electrical conductivity between the ruthenium surface to which it is bonded and the bridge molecule that is linked to the ruthenium electrode. In various embodiments, a bifunctional linker comprises a structure configured to improve electrical conductivity between a bridge molecule and the ruthenium electrode to which it is linked. The transmission of a signal across this bridging molecule can be tuned by controlling the electronics of the bifunctional linker itself through the inclusion of aromatic groups, and/or various R substituent groups than can tune the electronic structure of the linkage.

It should be understood that the present disclosure illustrates and describes a single bifunctional linker molecule bonding to a ruthenium surface. This is simply for purposes of illustration and for the sake of clarity and comprehension. When a ruthenium surface is immersed in, or otherwise exposed to, a solution of a bifunctional linker as described herein, wherein the bifunctional linker has a reactive diazonium, diazo or carbene group, the ruthenium surface is essentially “passivated,” meaning it is substantially coated with a film of bifunctional linker molecules, not just “one” bifunctional linker molecule. Such passivation processes may involve electrochemical processes, as discussed herein, and are sometimes referred to as “spontaneous grafting.” However, portions of a ruthenium surface may be masked, such as with a patterned PMMA layer, preventing bonding of the bifunctional linkers to the ruthenium under the masked portions, thus reducing the size of passivated areas onto which a bridge molecule, (and preferably only a single bridge molecule), may bind. Further, by switching potential during the electrochemical passivation, functionalization of a first ruthenium electrode followed by functionalization of a second ruthenium electrode in a pair of electrodes, can be stepwise and controlled.

In various embodiments, a bifunctional linker usable to bond various molecules such as biopolymeric bridge molecules or synthetic organic bridge molecules to ruthenium is characterized by the general structure:

A-L-Z, wherein,

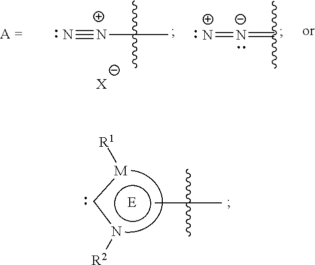

X=Cl—, Br—, I—, BF₄—, ClO₄—, SO₄²⁻, or another suitable anion;

Z=—CO₂H, —NH₂, —OH, —OC(O)C(CH₃)₂—Br, —CH═CH₂, —SH, —C≡CH or N₃; and

L is a bivalent tether of any length and combination of carbon and heteroatoms; wherein,

M is N or S, E is a heterocycle selected from imidazole, imidazoline, thiazole, or triazole; and R¹ and R² are independently selected from an electron pair, H, an aliphatic substituent, or an aryl substituent.

In various embodiments, the “A-end” of the bifunctional linker is configured to bond to the ruthenium surface whereas the “Z-end” of the bifunctional linker is configured to enter into conjugation with one end of a bridge molecule, such as through a condensation reaction to form an amide or ester.

In various embodiments, bifunctional linkers of structure A-L-Z as set forth above are both activated and unprotected. For example, a diazonium salt may begin as amine, in which case the bifunctional linker having the amine moiety may be treated with sodium nitrite in hydrochloric acid to generate the diazonium chloride for reaction with a ruthenium surface. In another example, the heterocyclic carbene may begin as an imidazolium salt requiring deprotonation in alkali to generate the reactive carbene for bonding to ruthenium. Also, for Z=—CO₂H, —NH₂, —OH, or —SH, these Z functional groups can be protected (e.g., as esters, carbamates, etc.), in which case the Z group would be deprotected prior to reaction with one end of the bridge molecule.

In various embodiments, bifunctional linkers having a diazonium salt functionality are used to generate a carbon radical or cation that then bonds to a ruthenium surface. Aryl diazonium salts are generally more common than aliphatic diazonium salts. Therefore, in various embodiments, the tether L in the bifunctional linker A-L-Z may comprise a phenyl ring at the “A end,” and that phenyl ring can carry the diazonium salt substituent as the A group.

In various embodiments, the bivalent tether L comprises -G-CH—; -G-(CH₂)_{y}—; or -G-(CH₂CH₂O)_{y}—, wherein y=1 to 25 and G is a covalent bond or an aryl linkage. In various embodiments, G is aryl such that one end of the bivalent tether L terminates in an aryl group, such as phenyl. As discussed below, a terminal aryl group on the bivalent tether L can carry a diazonium salt substituent. If present within L, G can be any aryl linkage, and can be denoted as —Ar— to show the connection between A and L.

In various embodiments, a bifunctional linker usable to bond molecules to ruthenium comprises the general structure:

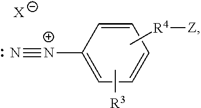

wherein, R³ represents any number (up to four) or type of substituents on the phenyl ring, including H, halide, nitro, methoxy, trifluoromethyl, alkyl or aryl, and R⁴ is a covalent bond or a bivalent moiety used to extend the length of L. When R⁴ is a covalent bond and all instances of R³ are H, then L in the above structure of A-L-Z is simply —C₆H₄—. So, the bifunctional linker may be as simple as benzenediazonium salt (tetrafluoroborate, for example) with Z as the single substituent in the o-, m- or p-position. In various embodiments, R⁴ is —(CH₂)_{y}—, wherein y=1 to 25, and wherein any number of intervening heteroatoms such as —O— may be present. In various embodiments, R⁴ is polyethylene glycol. In various embodiments, the length of R⁴ may be adjusted to accommodate a particular length bridge molecule intended to span a nanogap between electrodes in a pair of electrodes. In related embodiments, the phenyl ring may be replaced with any other aryl moiety, such as naphthyl.

In various embodiments, a solution of a bifunctional linker with A=diazonium salt may be reacted with a ruthenium surface under electrochemical reduction condition to bond the bifunctional linker to the ruthenium, as per the following reaction scheme that likely involves an intermediate aryl radical:

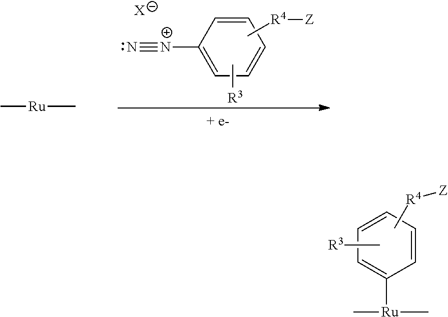

In the above scheme, Z is then available for conjugation to one end of a bridge molecule in the construction of a molecular sensor. If Z was previously protected, the protecting group can be removed such that the deprotected Z group can enter into a conjugation reaction with one end of a bridge molecule.

In various embodiments, the A group of a bifunctional linker A-L-Z comprises a diazo substituent bonded to a carbon atom in the tether L. A diazo group can react to form a carbene, which then is available to bond to a ruthenium surface. In general, a carbene can be generated from a diazo compound by a number of methods, including photochemical (i.e., photolytic decomposition), thermal decomposition, and metal-catalyzed decomposition.

In various embodiments, a solution of a bifunctional linker with A=diazo may be reacted with a ruthenium surface under photolytic, thermal or metal-catalyzed degradation conditions to bond the bifunctional linker to the ruthenium, as per the following reaction scheme that likely involves an intermediate carbene:

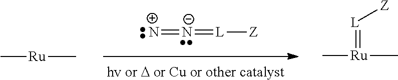

In the above scheme, Z is then available for conjugation to one end of a bridge molecule in the construction of a molecular sensor. If Z was previously protected, the protecting group can be removed such that the deprotected Z group can enter into a conjugation reaction with one end of a bridge molecule.

In various embodiments, the A group in a bifunctional linker comprises a heterocyclic carbene precursor. For this purpose, imidazolium salts may be used that can be deprotonated with strong alkali to produce a carbene that bonds to the ruthenium surface.

In various embodiments, the A group in a bifunctional linker of structure A-L-Z comprises an imidazolium salt, wherein the L-Z linkage is bonded to the imidazoline ring at either the 4- or 5-, and wherein the imidazolium ring has a substituent on the quaternary nitrogen N1 and optionally a substituent on the uncharged nitrogen N2. Further, the imidazolium ring may include an additional substituent in the remaining C-position not occupied by the L-Z linkage, (i.e., either the 4- or 5-position).

In various embodiments, a bifunctional linker of structure A-L-Z wherein A is an imidazolium ring can be reacted with a ruthenium surface in the presence of a strong base to deprotonate the hydrogen in the 2-position, as per the following reaction scheme:

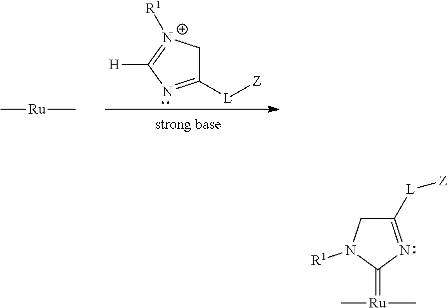

It should be understood that the above reaction scheme only illustrates embodiments of a bifunctional linker wherein A=an imidazoline salt, rather than other heterocyclic carbene precursors such as a thiazole. Further, the counterion to the imidazolium salt can be any practical anion, such as Cl—, Br—, I—, BF₄—, ClO₄—, SO₄²⁻, or another suitable anion. In the above scheme, Z is then available for conjugation to one end of a bridge molecule in the construction of a molecular sensor. If Z was previously protected, the protecting group can be removed such that the deprotected Z group can enter into a conjugation reaction with one end of a bridge molecule.

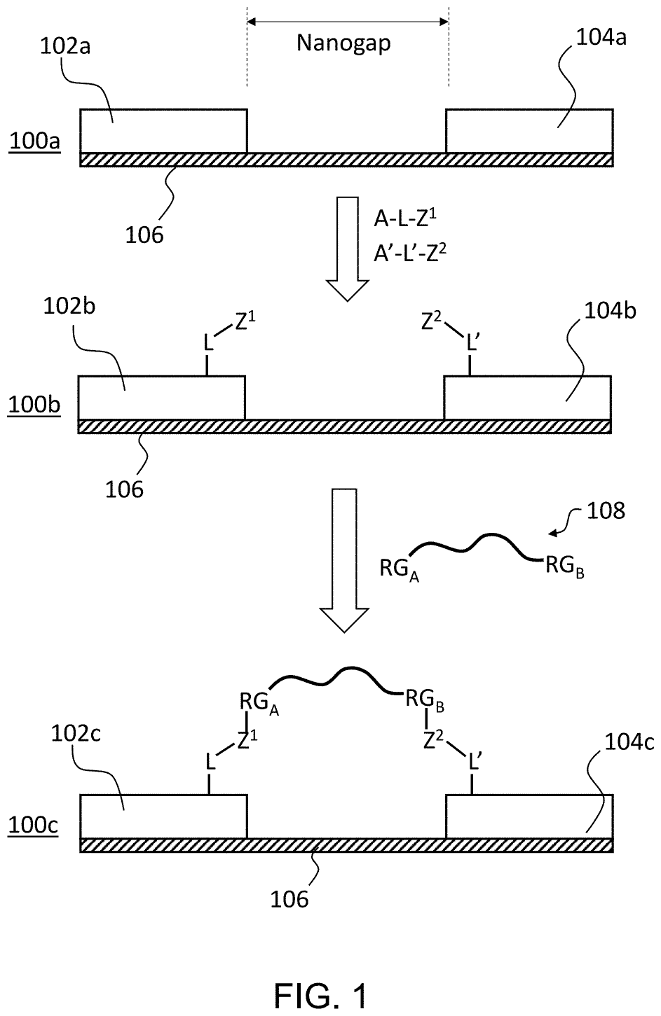

With reference now to FIG. 1, a process for forming a sensor circuit comprising covalent bonding to ruthenium electrodes is illustrated.

In FIG. 1, sensor substructure 100a comprises two spaced apart ruthenium electrodes 102a and 104a on a substrate 106. The first ruthenium electrode 102a is spaced-apart from the second ruthenium electrode 104a by a nanogap as shown. The nanogap may be from about 1 nm to about 1 μm, with a preferred range of about 1 nm to about 50 nm. The substrate 106 may comprise Si or Si with an intervening SiO₂ between the Si substrate and the electrodes. The ruthenium electrodes may comprise nanoscale deposits of ruthenium on the substrate 106. Not shown in FIG. 1 are other circuit elements that may be connected to the first ruthenium electrode 102a and/or the second ruthenium electrode 104a. Such circuitry elements may include a voltage or current source. There may also be a third electrode, not illustrated, such as a buried gate electrode positioned between and under the electrodes 102a and 104a. The additional circuit elements may be necessary for applying a potential across the electrodes to direct electrochemical passivation of one electrode over the other. Then, by switching the polarity of the potential, the other electrode in the pair of electrodes may be passivated with the same or different bifunctional linker. In particular, a negative (−) polarized electrode may participate in electrochemical reduction of diazo compounds. Further, one electrode may be temporarily masked by a removable mask layer such that only the unmasked electrode is passivated with the bifunctional linker.

In transitioning between substructure 100a and substructure 100b, each of two bifunctional linkers are used to passivate each of the ruthenium electrodes in stepwise fashion. In this schematic, the bifunctional linker A-L-Z¹ is used to passivate the first ruthenium electrode 102a, converting it to a passivated first ruthenium electrode 102b. The nature of the A and A′ groups dictate the reaction conditions. For example, if A is a diazonium salt, then an electrochemical reduction process may be used to generate a carbon radical on L that binds to the ruthenium. In a separate step, the bifunctional linker A′-L′-Z² is used to passivate the second ruthenium electrode 104a, converting it to a passivated second ruthenium electrode 104b. A-L-Z¹ and A′-L′-Z² can be identical substances if a flip in polarity is used to selectively passivate one electrode over the other, or if one electrode is temporarily masked and blocked from passivation. As discussed above, Z¹ and Z² may be protected by a protecting group “P” such that each Z group does not interfere in the bonding of A and A′ to the ruthenium surface. In certain variations, A and A′ may be the same, and/or Z¹ and Z² may be the same functional group but configured with different protecting groups, or Z¹ and Z² may be different. In various embodiments, L and L′ may the same or different bivalent tethers.

The next step in forming a sensor circuit 100c is to bond the bridge molecule 108 to each of the ruthenium electrodes such via each of the placed bifunctional linkers that the bridge molecule 108 spans the nanogap. As mentioned, a bridge molecule 108 comprises a first end having a first reactive group RG_{A} at or near the first end, and a second end having a second reactive group RG_{B} at or near the second end. For self-assembly to be at least somewhat efficient, the average distance between RG_{A} and RG_{B} (recognizing continual conformation changes in the molecule and thus a dynamically changing distance between the RG_{A} and RG_{B} groups) is somewhat similar to the distance between Z¹ and Z² groups. However, unless portions of the ruthenium electrodes 102a and 104a were masked, the majority of the exposed surfaces of the electrodes may be coated with a film comprising a plethora of bifunctional linker molecules bonded to ruthenium, in which case the average distance between RG_{A} and RG_{B} should be greater than the nanogap distance, and not so short as to promote unproductive loops on the same electrode.

In various embodiments, either or both RG_{A} and RG_{B} groups may be protected, as well as either of both Z¹ and Z². Conjugating the bridge molecule then becomes a matter of selectively deprotecting RG_{A} and Z¹ and performing the conjugation reaction between the two, and then deprotecting RG_{B} and Z² and performing the conjugation reaction between those two. As mentioned, these conjugation reactions may be condensation reactions forming amides or esters, or etherification, or a form of “click-chemistry,” such as between an azide and an alkyne to produce a triazole or any one of a diverse set of inverse electron demand Diels-Alder reactions (iEDDA).

In instances where the bridge molecule 108 comprises a polypeptide, the bridge molecule 108 may naturally have an RG_{A} comprising a carboxylic acid moiety and an RG_{B} comprising an amino moiety. In this case, it may not be necessary to provide protecting groups, as Z¹ could be an amino group to form an amide with the RG_{A} carboxylic acid moiety and Z² could be a carboxylic acid group to form another amide with the RG_{B} amino moiety.

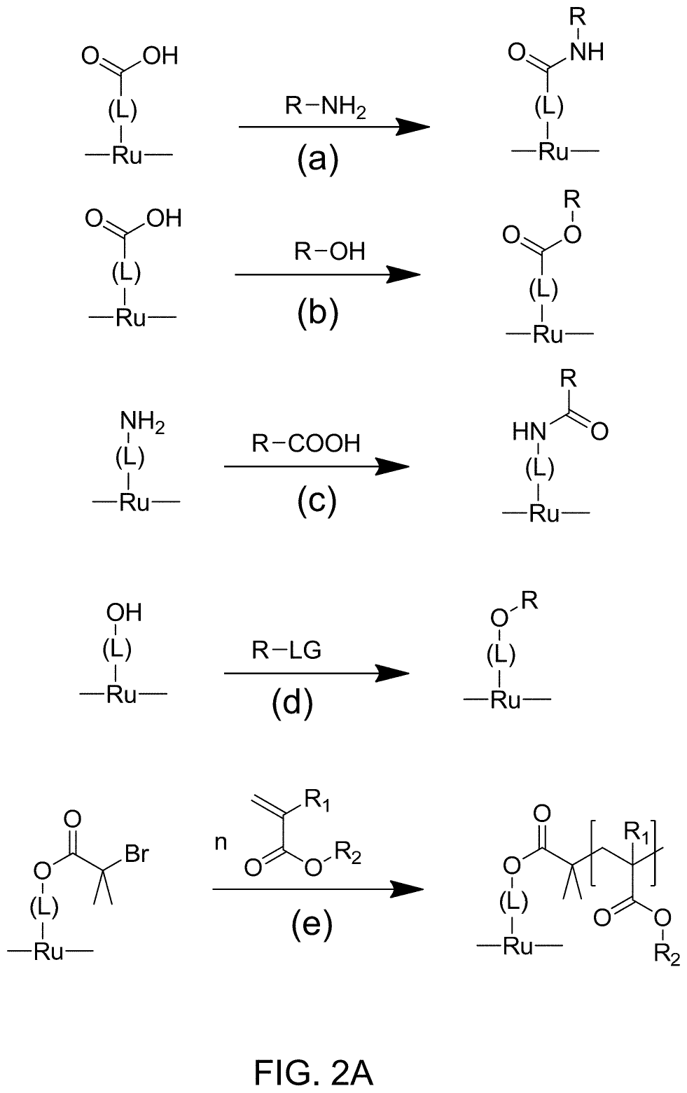

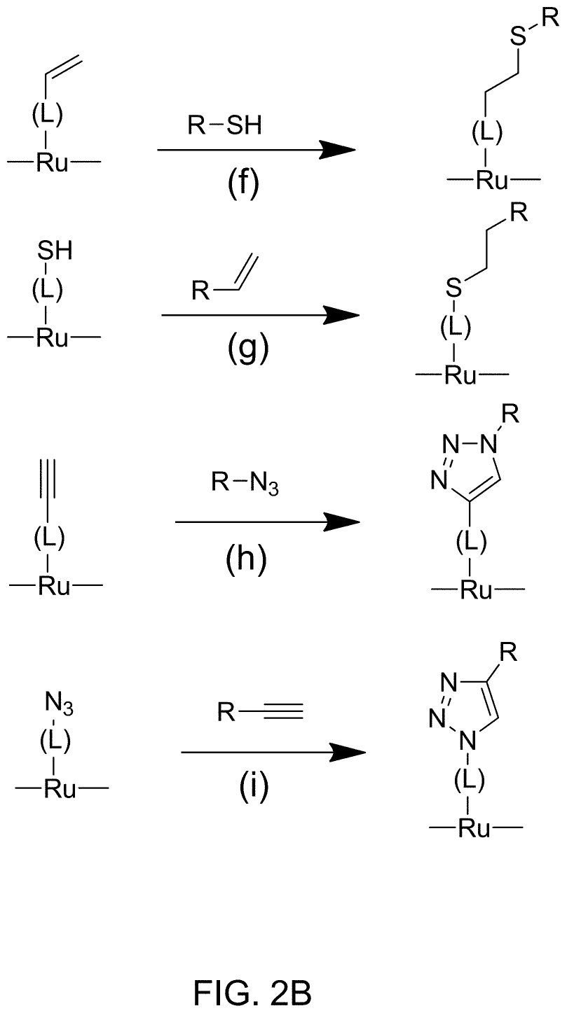

FIGS. 2A and 2B set forth non-limiting examples of unprotected Z groups once the bifunctional linker A-L-Z is bonded to ruthenium. For simplicity, only examples are shown wherein A was either a diazonium salt or a diazo group, liberated as nitrogen N₂ in the reaction that provided the bond to ruthenium. In various embodiments where A comprises a heterocyclic moiety capable of forming a carbene, (such as an imidazoline salt), the heterocycle is not shown in the examples for clarity, and would necessarily be structurally between L and the ruthenium surface “—R—” since only a proton is removed in the conversion of the carbene precursor to the carbene and not the entire heterocycle.

With reference now to FIG. 2A, reaction (a) comprises a carboxylic Z group from the bifunctional linker A-L-Z reacting with an amino group on the end of the bridge molecule to form an amide.

Reaction (b) comprises a carboxylic Z group from the bifunctional linker A-L-Z reacting with a hydroxyl group on the end of the bridge molecule to form an ester.

Reaction (c) comprises an amino Z group from the bifunctional linker A-L-Z reacting with a carboxylic acid group on the end of the bridge molecule to form an amide.

Reaction (d) comprises a hydroxyl Z group from the bifunctional linker A-L-Z displacing a leaving group LG on the end of the bridge molecule in an S_{N}1 or S_{N}2 reaction to form an ether.

Reaction (e) comprises a Z group configured with a tertiary bromide that can form a carbon radical to react with the alkene portion of an α, β-unsaturated ester moiety on the end of the bridge molecule to form a C—C linkage.

FIG. 2B sets forth further examples of Z groups configured on the bifunctional linkers A-L-Z,

With reference now to FIG. 2B, reaction (f) comprises an alkene Z group from the bifunctional linker A-L-Z reacting with a thiol group on the end of the bridge molecule to form a sulfide.

Reaction (g) comprises a thiol Z group from the bifunctional linker A-L-Z reacting with an alkene group on the end of the bridge molecule to form a sulfide.

Reaction (h) comprises an alkyne Z group from the bifunctional linker A-L-Z reacting with an azide group on the end of the bridge molecule as a click-chemistry pair to form a triazole.

Reaction (i) comprises an azide Z group from the bifunctional linker A-L-Z reacting with an alkyne group on the end of the bridge molecule as a click-chemistry pair to form a triazole.

The non-limiting examples of Z groups in FIGS. 2A and 2B show the many possible conjugation reactions that can be employed in conjugating the Z group from the bifunctional linker A-L-Z to one end of a bridge molecule.

### EXAMPLES

In express examples, three different bifunctional linker molecules were tested in passivation of ruthenium surfaces, namely t-butyl diazoacetate, 4-diazo-3-methoxydiphenylamine sulfate, and 1,3-dimesityl-1H-imidazol-3-ium tetrafluoroborate, as illustrated below:

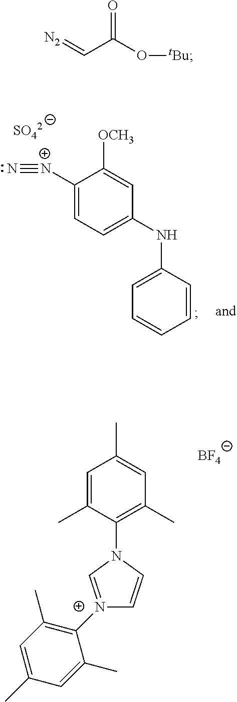

t-Butyl diazoacetate fits the general bifunctional linker formula A-L-Z when A is —N₂, L is —CH— and Z is —CO₂H protected as a t-butyl ester. Thus, t-butyl diazo acetate can be used to passivate a ruthenium surface with a film of acetate molecules bonded to the ruthenium. 4-Diazo-3-methoxydiphenylamine sulfate and 1,3-dimesityl-[1H]-imidazol-3-ium tetrafluoroborate are used to demonstrate the concept of bonding a reactive carbene to ruthenium since these two molecules do not readily fit the bifunctional linker formula A-L-Z.

Ruthenium substrates were prepared by sputtering 200 nm thick Ru layer onto a Si wafer. These wafers were then prepared for functionalization by testing both chemical and plasma treatment processes. Preparation processes investigated were, no treatment, argon plasma treatment at 100 W for 3 minutes, 18 hour exposure to 2M citric acid, and 18 hour exposure to 2M sodium borohydride. After pre-treatment, the surfaces were exposed to the following chemical reagents for surface functionalization: 15% t-butyl diazoacetate in toluene, 5% 4-diazo-3-methoxydiphenylamine sulfate in ethylene glycol, and 15% 1,3-dimesityl-[1H]-imidazol-3-ium tetrafluoroborate in acetonitrile. The reactions proceeded at room temperature for 18 hours, and the surfaces were rinsed clean.

Surface functionalization was tracked by measuring the contact angle of a water droplet on ruthenium surfaces before and after functionalization.

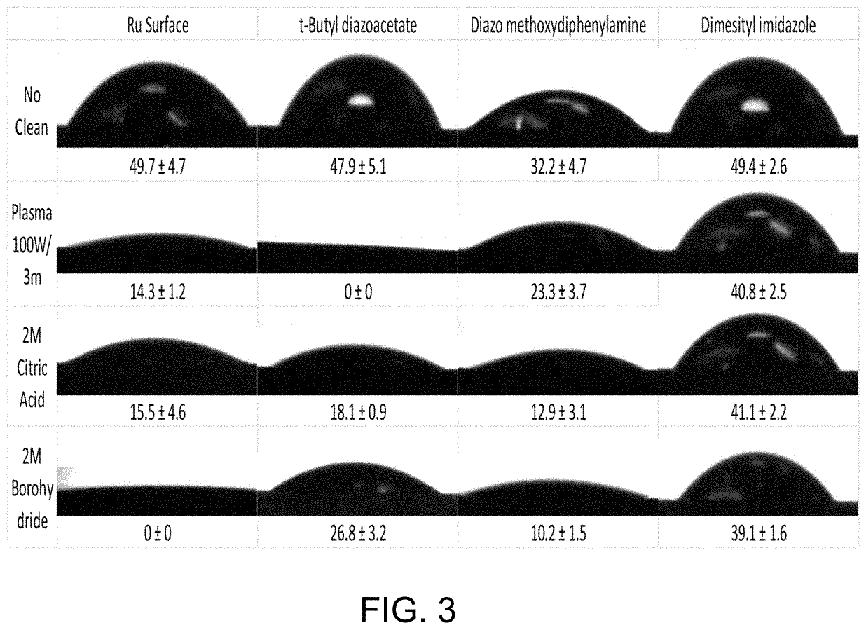

As shown by the contact angles in FIG. 3, changes in wettability show a change in surface properties based on the functionalization chemistry.

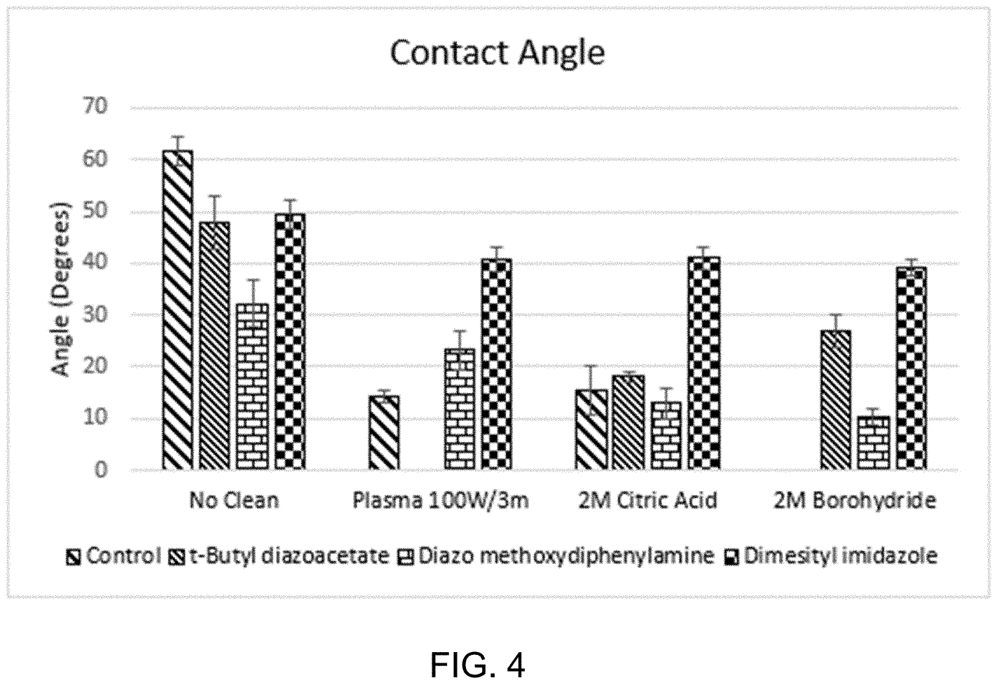

FIG. 4 sets forth the change in contact angle for no functionalization versus varied chemical functionalization for different surface pretreatment conditions. It is clear from the results that pretreatment of the ruthenium surface improves bonding of each of the three test compounds to the ruthenium surface, and that there is little difference between the types of pretreatment conditions used. No conclusions are made regarding the effect of chemical structure on bonding and passivation since each of the test compounds differ in hydrophobicity.

Additional Aspects

In various embodiments, bifunctional linkers are disclosed that find use in bonding bridge molecules to ruthenium electrodes in sensor manufacturing.

In various embodiments, methods of making molecular sensors comprises a step of passivating an electrode surface with a bifunctional linker and then conjugating the attached linker to the molecule used in the molecular sensor.

In various embodiments, a bifunctional linker comprises the structure A-L-Z, wherein A, L and Z are defined as per above. In various embodiments, A is reactive toward ruthenium such that the bifunctional linker can be covalently bonded at one end to ruthenium. In various embodiments, Z is configured to enter into condensation or “click-chemistry” reactions with a functionalized end of a molecule needed in a molecular sensor. In various embodiments, the molecule conjugated to the bifunctional linker comprises a bridge molecule.

In various embodiments, methods for making sensor circuits are disclosed. The method comprises:

depositing a pair of ruthenium electrodes on a substrate, the pair of ruthenium electrodes comprising a first ruthenium electrode and a second ruthenium electrode spaced-apart from the first ruthenium electrode by a nanogap;

exposing the first ruthenium electrode to a bifunctional linker having a structure A-L-Z¹ to functionalize the first ruthenium electrode with a plurality of exposed Z¹ groups;

conjugating at least one exposed Z¹ group to a first reactive group RG_{A} configured at or near a first end of a bridge molecule, the bridge molecule further comprising a second reactive group RG_{B} configured at a second end of the bridge molecule;

exposing the second ruthenium electrode to a bifunctional linker having a structure A′-L′-Z² to functionalize the second ruthenium electrode with a plurality of exposed Z² groups; and

conjugating at least one exposed Z² group to the second reactive group RG_{B} configured at or near the second end of the bridge molecule.

In various embodiments, a sensor circuit is disclosed. The sensor circuit comprises:

a pair of ruthenium electrodes comprising a first ruthenium electrode and a second ruthenium electrode spaced-apart from the first ruthenium electrode by a nanogap;

a bridge molecule comprising a first reactive group RG_{A} configured at or near a first end, and a second reactive group RG_{B} configured at a second end, the bridge molecule electrically wired to each of the first and second ruthenium electrodes and spanning the nanogap;

wherein the first reactive group RG_{A} is conjugated to a first reactive group Z¹ covalently bonded to the first ruthenium electrode through a first tether L, and the second reactive group RG_{B} is conjugated to a second reactive group Z² covalently bonded to the second ruthenium electrode through a second tether L′.

In the detailed description, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected, coupled or the like may include permanent (e.g., integral), removable, temporary, partial, full, and/or any other possible attachment option. Any of the components may be coupled to each other via friction, snap, sleeves, brackets, clips or other means now known in the art or hereinafter developed. Additionally, any reference to without contact (or similar phrases) may also include reduced contact or minimal contact.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

1. A sensor circuit comprising:
a pair of ruthenium electrodes comprising a first ruthenium electrode and a second ruthenium electrode spaced-apart from the first ruthenium electrode by a nanogap; and
a bridge molecule comprising a first reactive group RG_{A} configured at or near a first end, and a second reactive group RG_{B} configured at a second end, the bridge molecule electrically wired to each of the first and second ruthenium electrodes and spanning the nanogap;
wherein the first reactive group RG_{A} is conjugated to a first reactive group Z¹ covalently bonded to the first ruthenium electrode through a first bivalent tether L, and the second reactive group RG_{B} is conjugated to a second reactive group Z² covalently bonded to the second ruthenium electrode through a second bivalent tether L′.

2. The sensor circuit of claim 1, wherein the bridge molecule comprises a polypeptide, a protein, a protein fragment, a protein alpha-helix, DNA, RNA, a single-stranded oligonucleotide, a double-stranded oligonucleotide, a peptide nucleic acid duplex, a peptide nucleic acid-DNA hybrid duplex, an antibody, an antibody Fab binding domain, a carbon nanotube, a graphene-like polycyclic aromatic nanoribbon, other natural polymers, or (poly)thiophene.

3. The sensor circuit of claim 1, wherein RG_{A} and RG_{B} are independently selected from —CO₂H, —NH₂, —OH, —SH, —CH═CH₂, —C≡CH, and —N₃.

4. The sensor circuit of claim 1, wherein L and L′ are independently selected from —CH—; —(CH₂)_{y}—; or —(CH₂CH₂O)_{y}—, wherein y=1 to 25.

5. The sensor circuit of claim 1, wherein L further comprises a phenyl ring or substituted phenyl ring covalently bonded to the first ruthenium electrode.

6. The sensor circuit of claim 1, wherein L′ further comprises a phenyl ring or substituted phenyl ring covalently bonded to the second ruthenium electrode.

7. A bifunctional linker configured to covalently bond a molecule to a ruthenium surface, the bifunctional linker molecule having a structure, A-L-Z, wherein: 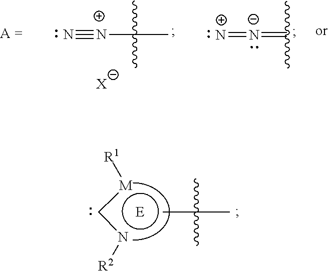
X=Cl—, Br—, I—, BF₄—, ClO₄—, or (SO₄²⁻)_{1/2};
Z=—CO₂H, —NH₂, —OH, —OC(O)C(CH₃)₂—Br, —CH═CH₂, —SH, —C≡CH or N₃;
L is a bivalent tether selected from -G-CH—; -G-(CH₂)_{y}—; or -G-(CH₂CH₂O)_{y}—, wherein y=1 to 25 and G is an optional aryl linkage —Ar—;
M=N or S, and E is a heterocycle selected from imidazole, imidazoline, thiazole, or triazole; and
R¹ and R² are independently selected from an electron pair, H, an aliphatic substituent, or an aryl substituent.

8. The bifunctional linker of claim 7, wherein A is a diazonium salt and Z is —CO₂H, —NH₂, —OH, —OC(O)C(CH₃)₂—Br, —CH═CH₂, —SH, —C≡CH or N₃.

9. The bifunctional linker of claim 7, wherein A is a diazo group and Z is —CO₂H, —NH₂, —OH, —OC(O)C(CH₃)₂—Br, —CH═CH₂, —SH, —C≡CH or N₃.

10. The bifunctional linker of claim 7, wherein A is an imidazolium ring and Z is —CO₂H, —NH₂, —OH, —OC(O)C(CH₃)₂—Br, —CH═CH₂, —SH, —C≡CH or N₃.

11. A method of forming a sensor circuit, the method comprising:
depositing a pair of ruthenium electrodes on a substrate, the pair of ruthenium electrodes comprising a first ruthenium electrode and a second ruthenium electrode spaced-apart from the first ruthenium electrode by a nanogap;
exposing the first ruthenium electrode to a bifunctional linker having a structure A-L-Z¹ to functionalize the first ruthenium electrode with a plurality of exposed Z¹ groups;
conjugating at least one exposed Z¹ group to a first reactive group RG_{A} configured at or near a first end of a bridge molecule, the bridge molecule further comprising a second reactive group RG_{B} configured at a second end of the bridge molecule;
exposing the second ruthenium electrode to a bifunctional linker having a structure A′-L′-Z² to functionalize the second ruthenium electrode with a plurality of exposed Z² groups; and
conjugating at least one exposed Z² group to the second reactive group RG_{B} configured at or near the second end of the bridge molecule.

12. The method of claim 11, wherein:
A and A′ are independently, 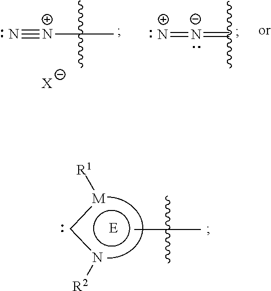
X=Cl—, Br—, I—, BF₄—, ClO₄—, or (SO₄²⁻)_{1/2};
Z¹ and Z² are independently selected from:
—CO₂H, —NH₂, —OH, —OC(O)C(CH₃)₂—Br, —CH═CH₂, —SH, —C≡CH, and N₃;
L and L′ are each a bivalent tether independently selected from -G-CH—; -G-(CH₂)_{y}—;
or -G-(CH₂CH₂O)_{y}—, wherein y=1 to 25 and G is an optional aryl linkage —Ar—;
M=N or S, and E is a heterocycle selected from imidazole, imidazoline, thiazole, or triazole; and
R¹ and R² are independently selected from an electron pair, H, an aliphatic substituent, or an aryl substituent.

13. The method of claim 11, further comprising a step of polarizing the first ruthenium electrode prior to exposing the first ruthenium electrode to a bifunctional linker having a structure A-L-Z′ such that the bifunctional linker covalently bonds to the first ruthenium electrode via electrochemical reduction.

14. The method of claim 11, further comprising a step of polarizing the second ruthenium electrode prior to exposing the second ruthenium electrode to a bifunctional linker having a structure A′-L′-Z² such that the bifunctional linker covalently bonds to the first ruthenium electrode via electrochemical reduction.

15. The method of claim 11, wherein the bridge molecule comprises a polypeptide, and RG_{A} and RG_{B} are independently selected from —CO₂H, —NH₂, —OH, —SH, —CH═CH₂, —C≡CH, and —N₃.
